(19) United States
(12) Patent Application Publication
(10) Pub. No.: US 2023/0274424 A1
(43) Pub. Date: Aug. 31, 2023

(54) APPARTUS AND METHOD FOR QUANTIFYING LESION IN BIOMETRIC IMAGE

(71) Applicant: CONNECTEVE CO., Ltd., Seoul (KR)

(72) Inventors: Doo Hyun HWANG, Seoul (KR); Du Hyun RO, Seoul (KR)

(73) Assignee: CONNECTEVE CO., Ltd., Seoul (KR)

(21) Appl. No.: 18/114,913

(22) Filed: Feb. 27, 2023

(30) Foreign Application Priority Data
Feb. 28, 2022 (KR) 10-2022-0025789

(51) Int. Cl. G06T 7/00; G06T 7/62

(52) U.S. Cl. CPC G06T 7/0012; G06T 7/62; G06T 2207/30096; G06T 2207/20081

(57) ABSTRACT

Provided are a computing device and methods for quantifying a lesion in a biometric image. In certain aspects, disclosed a method including the steps of: extracting a lesion information in a plurality of first biometric images from each of the plurality of first biometric images three-dimensionally photographed of an object based on a machine learning model; generating a plurality of second biometric images in which a region of the lesion information, by performing image processing on each of the plurality of first biometric images; and calculating a volume of the lesion quantitatively using the region of the lesion information.

### A. TECHNICAL FIELD

The present disclosure relates to quantifying lesions in a biometric image, and more particularly, to a method and apparatus capable of quantifying lesions in a 3-dimensional tomographic biometric image using a machine learning model and tracking and observing the lesions based on quantified.

### B. DESCRIPTION OF THE RELATED ART

With development of artificial intelligence learning models, many machine learning models are being used to read medical images. For example, the machine learning models such as Convolutional Neural Networks (CNN), Deep Neural Networks (DNN), Recurrent Neural Networks (RNN), and Deep Belief Networks (DBN) are being applied to detect, classify, and characterize the medical images.

A machine learning model is used to support image reading (finding/diagnosis) to predict a test subject's disease. More specifically, a medical image of a person to be examined is acquired, attribute information is extracted based on a machine learning model and is provided to a diagnoser. On the basis of attribute information, the diagnoser predicts a disease or determines a progression of a disease. At this time, the attribute information includes various information included in the medical image.

On the other hand, bone tumor treatment in the medical field is a process that should be performed after treatment for treatment evaluation and recurrence determination. In order to evaluate the treatment and determine whether the disease will recur or not, Bone tumors shown in a 2-dimensional image (e.g., X-ray image) or a 3-dimensional image (e.g., CT, MRI image) are identified and judged based on the patient's medical history, age, clinical symptoms, anatomical location of the lesion, etc. As an example, when a bone tumor treatment is evaluated based on a 3D image, the evaluation depends on qualitative and subjective interpretations of changes in T1 and T2 relaxation characteristics of a lesion (tumor), which is the reason that medical staff who are not accustomed to interpreting images of bone tumors are bound to be more inaccurate in postoperative treatment and evaluation of recurrence.

In particular, according to the response evaluation criterion of the World Health Organization (WHO) for standardization of recording and reporting of treatment effects of solid tumors, about 3-dimensional medical images used for detailed judgment of tumor treatment, selecting the 2-dimensional cross section where the tumor looks the largest in the 3-dimensional image before treatment and measuring the long axis and the longest short axis perpendicular to the long axis of the tumor were carried out. And, after treatment, the method to measure the increase or decrease in the long axis and short axis of the tumor was carried out.

However, since bone tumors essentially have a three-dimensional structure, evaluation of treatment and determination of recurrence after treatment by the conventional method have substantially inaccuracies. As such, there is a need for quantitative measurement of the size or volume of the lesion as well as the exact location of the lesion (tumor) after tumor treatment, in order to increase the accuracy of evaluation of treatment and determination of recurrence.

### SUMMARY OF THE DISCLOSURE

In one aspect of the present disclosure, a computing device comprises a processor; and a memory that is communicatively coupled to the processor and stores one or more sequences of instructions, which when executed by the processor causes steps to be performed comprising: extracting a lesion information in a plurality of first biometric images from each of the plurality of first biometric images three-dimensionally photographed of an object based on a machine learning model; generating a plurality of second biometric images in which a region of the lesion information, by performing image processing on each of the plurality of first biometric images; and calculating a volume of a lesion quantitatively using the region of the lesion information.

Desirably, the lesion information may include at least one of a size of the lesion and a location of the lesion.

Desirably, the lesion may be a solid tumor including a bone tumor.

Desirably, the processor may perform image processing on the lesion information in the plurality of second biometric to generate a plurality of third biometric images in which only the region of the lesion information is visualized.

### BRIEF DESCRIPTION OF THE DRAWINGS

FIG. 1 shows a schematic diagram of an illustrative apparatus for quantifying a lesion in a biometric image according to embodiments of the present disclosure.

FIG. 2 is a schematic diagram of an illustrative system for quantifying a lesion in a biometric image according to embodiments of the present disclosure.

FIG. 3 shows a block diagram of a processor for identifying a lesion in a biometric image according to embodiments of the present disclosure.

FIG. 4 is a diagram illustratively showing a process of extracting attribute information from tomographic first biometric images and generating second biometric images by a computing device according to an embodiment of the present disclosure.

FIG. 5 is a diagram illustratively showing label information areas displayed on second biometric images generated by a computing device according to an embodiment of the present disclosure.

FIG. 6 is a diagram illustrating a third biological image visualizing a label information area in a second biological image generated by a computing device according to an embodiment of the present disclosure.

FIG. 7 is a flowchart illustrating a method for quantifying a lesion in a biometric image according to an exemplary embodiment of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices, and wireless connections.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity because of referring to performing machine learning by a processing module such as a processor, a CPU, an application processor, micro-controller, so on.

An “image” is defined as a reproduction or imitation of the form of a person or thing, or specific characteristics thereof, in digital form. An image can be, but is not limited to, a JPEG image, a PNG image, a GIF image, a TIFF image, or any other digital image format known in the art. “Image” is used interchangeably with “photograph”.

A “feature(s)” is defined as a group of one or more descriptive characteristics of subjects. A feature can be a numeric attribute.

The terms “comprise/include” used throughout the description and the claims and modifications thereof are not intended to exclude other technical features, additions, components, or operations.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

The embodiments described herein relate generally to diagnostic medical images. Although any type of medical image can be used, the disclosed methods, systems, apparatuses and devices can also be used with medical images of other ocular structures, or any other biological tissues image of which can support the diagnosis of a disease condition. Furthermore, the methods disclose herein can be used with a variety of imaging modalities including but not limited to: computed tomography (CT), magnetic resonance imaging (MRI), computed radiography, magnetic resonance, angioscopy, optical coherence tomography, color flow Doppler, cystoscopy, diaphanography, echocardiography, fluorescein angiography, laparoscopy, magnetic resonance angiography, positron emission tomography, single photon emission computed tomography, x-ray angiography, nuclear medicine, biomagnetic imaging, culposcopy, duplex Doppler, digital microscopy, endoscopy, fundoscopy, laser, surface scan, magnetic resonance spectroscopy, radio graphic imaging, thermography, and radio fluroscopy.

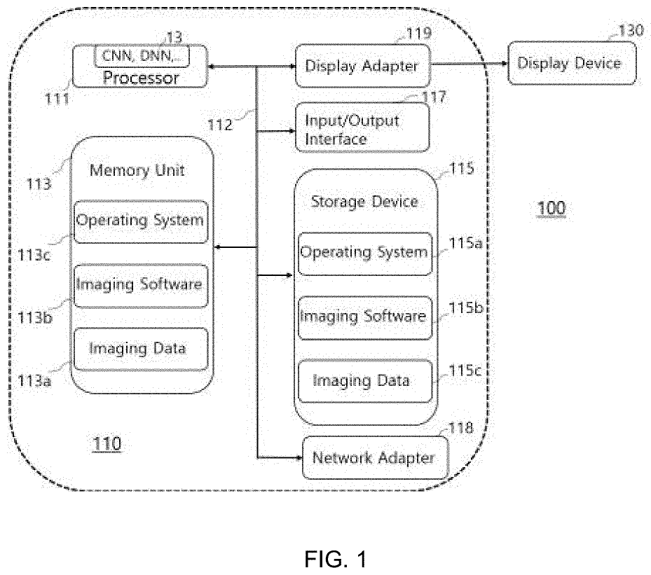

FIG. 1 shows a schematic diagram of an illustrative apparatus for quantifying a lesion in a biometric image according to embodiments of the present disclosure.

As depicted, the apparatus 100 may include a computing device 110 and a display device 130. In embodiments, the computing device 110 may include, but is not limited thereto, one or more processor 111, a memory unit 113, a storage device 115, an input/output interface 117, a network adapter 118, a display adapter 119, and a system bus 112 connecting various system components to the memory unit 113. In embodiments, the apparatus 100 may further include communication mechanisms as well as the system bus 112 for transferring information. In embodiments, the communication mechanisms or the system bus 112 may interconnect the processor 111, a computer-readable medium, a short range communication module (e.g., a Bluetooth, a NFC), the network adapter 118 including a network interface or mobile communication module, the display device 130 (e.g., a CRT, a LCD, etc.), an input device (e.g., a keyboard, a keypad, a virtual keyboard, a mouse, a trackball, a stylus, a touch sensing means, etc.) and/or subsystems.

In embodiments, the processor 111 is, but is not limited to, a processing module, a Computer Processing Unit (CPU), an Application Processor (AP), a microcontroller, a digital signal processor. In embodiments, the processor 111 may include an image filter such as a high pass filter or a low pass filter to filter a specific factor in the biometric image. In addition, in embodiments, the processor 111 may communicate with a hardware controller such as the display adapter 119 to display an operation of the apparatus 100 a user interface on the display device 130. In embodiments, the processor 111 may access the memory unit 113 and execute commands stored in the memory unit 113 or one or more sequences of instructions to control the operation of the apparatus 100. The commands or sequences of instructions may be read in the memory unit 113 from computer-readable medium or media such as a static storage or a disk drive, but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the processor 111 and may be loaded into the memory unit 113.

In embodiments, the system bus 112 may represent one or more of several possible types of bus structures, including a memory bus or memory controller, a peripheral bus, an accelerated graphics port, and a processor or local bus using any of a variety of bus architectures. For instance, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects(PCI), a PCI-Express bus, a Personal Computer Memory Card Industry Association (PCMCIA), Universal Serial Bus (USB) and the like. In embodiments, the system bus 112, and all buses specified in this description can also be implemented over a wired or wireless network connection.

A transmission media including wires of the system bus 112 may include at least one of coaxial cables, copper wires, and optical fibers. For instance, the transmission media may take a form of sound waves or light waves generated during radio wave communication or infrared data communication.

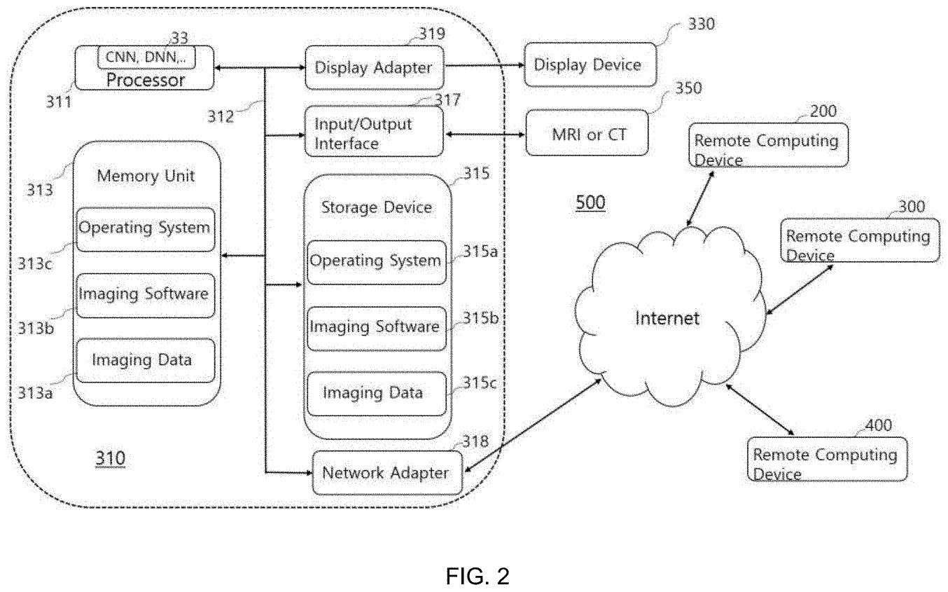

In embodiments, the apparatus 100 may transmit or receive the commands including messages, data, and one or more programs, i.e., a program code, through a network link or the network adapter 118. In embodiments, the network adapter 118 may include a separate or integrated antenna for enabling transmission and reception through the network link. The network adapter 118 may access a network and communicate with a remote computing devices 200, 300, 400 described in FIG. 2.

In embodiments, the network may be, but is not limited to, at least one of LAN, WLAN, PSTN, and cellular phone networks. The network adapter 118 may include at least one of a network interface and a mobile communication module for accessing the network. In embodiments, the mobile communication module may be accessed to a mobile communication network for each generation such as 2G to 5G mobile communication network.

In embodiments, on receiving a program code, the program code may be executed by the processor 111 and may be stored in a disk drive of the memory unit 113 or in a non-volatile memory of a different type from the disk drive for executing the program code.

In embodiments, the computing device 110 may include a variety of computer-readable medium or media. The computer-readable medium or media may be any available medium or media that are accessible by the computing device 100. For example, the computer-readable medium or media may include, but is not limited to, both volatile and non-volatile media, removable or non-removable media.

In embodiments, the memory unit 113 may store a driver, an application program, data, and a database for operating the apparatus 100 therein. In addition, the memory unit 113 may include a computer-readable medium in a form of a volatile memory such as a random access memory (RAM), a non-volatile memory such as a read only memory (ROM), and a flash memory. For instance, it may be, but is not limited to, a hard disk drive, a solid state drive, an optical disk drive.

In embodiments, each of the memory unit 113 and the storage device 115 may be program modules such as the imaging software 113b, 115b and the operating systems 113c, 115c that can be immediately accessed so that a data such as the imaging data 113a, 115a is operated by the processor 111.

In embodiments, the machine learning model 13 may be installed into at least one of the processor 111, the memory unit 113 and the storage device 115. The machine learning model 13 may be, but is not limited to, at least one of a deep neural network (DNN), a convolutional neural network (CNN) and a recurrent neural network (RNN), which are one of the machine learning algorithms.

For example, the Convolutional Neural Network (CNN) is a type of multilayer perceptrons designed to use minimal preprocessing. The Convolutional Neural Network (CNN) consists of one or several convolutional layers and general artificial neural network layers placed on top of the convolutional layers, and additionally utilizes weights and pooling layers. Thanks to the above structure, the Convolutional Neural Network(CNN) can fully utilize input data with a two-dimensional structure. Compared to other deep learning architectures, the Convolutional Neural Network (CNN) shows good performance in both video and audio fields. The Convolutional Neural Network(CNN) can also be trained via standard back-propagation. The Convolutional Neural Network(CNN) has the advantage of being easier to be trained than other feedforward artificial neural network techniques and using fewer parameters.

The Input/Output Interface 117 may receive a biometric image three-dimensionally photographed by a medical device such as CT or MRI from outside and provides the biometric image to the processor 111 to extract features based on the machine learning model 13. Images may be processed by the processor 111 and stored in the Memory Unit 113 or the Storage Device 115. In addition, the image-processed 3D biometric image may be provided to the remote computing devices 200, 300, 400 described in FIG. 2 through an internet network.

FIG. 2 is a schematic diagram of an illustrative system for quantifying a lesion in a biometric image according to embodiments of the present disclosure.

As depicted, the system 500 may include a computing device 310, a 3D medical image photographing device 350 such as MRI or CT and one and more remote computing devices 200, 300, 400. In embodiments, the computing device 310 and the remote computing devices 200, 300, 400 may be connected to each other through a network. The components 310, 311, 312, 313, 315, 317, 318, 319, 330 of the system 500 are similar to their counterparts in FIG. 1. In embodiments, each of remote computing devices 200, 300, 400 may be similar to the apparatus 100 in FIG. 1. For instance, each of remote computing devices 200, 300, 400 may include each of the subsystems, including the processor 311, the memory unit 313, an operating system 313c, 315a, an imaging software 313b, 315b, an imaging data 313a, 315c, a network adapter 318, a storage device 315, an input/output interface 317 and a display adapter 319. Each of remote computing devices 200, 300, 400 may further include a display device 330 and a camera 350. In embodiments, the system bus 312 may connect the subsystems to each other.

In embodiments, the computing device 310 and the remote computing devices 200, 300, 400 may be configured to perform one or more of the methods, functions, and/or operations presented herein. Computing devices that implement at least one or more of the methods, functions, and/or operations described herein may comprise an application or applications operating on at least one computing device. The computing device may comprise one or more computers and one or more databases. The computing device may be a single device, a distributed device, a cloud-based computer, or a combination thereof.

It shall be noted that the present disclosure may be implemented in any instruction-execution/computing device or system capable of processing data, including, without limitation laptop computers, desktop computers, and servers. The present invention may also be implemented into other computing devices and systems. Furthermore, aspects of the present invention may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

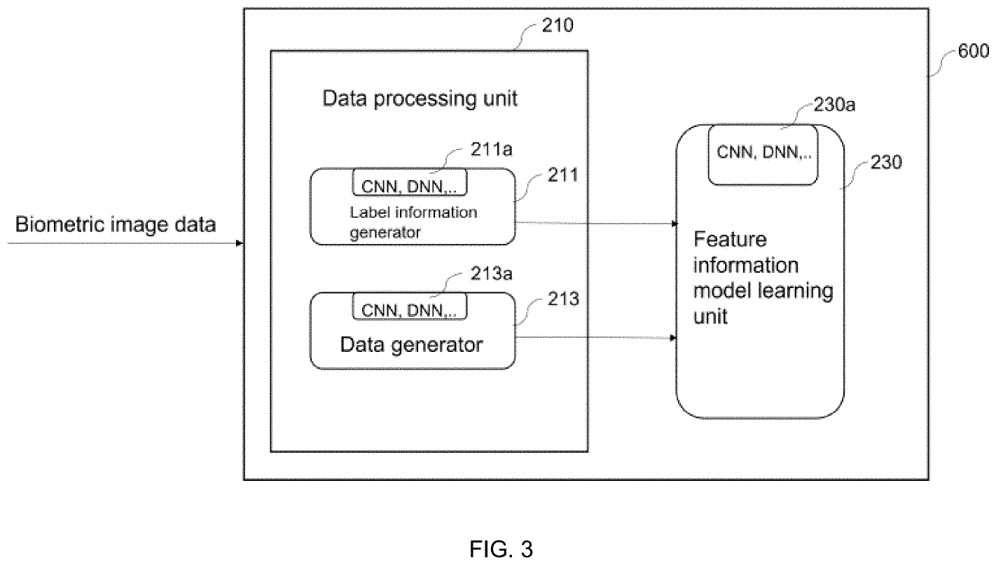

FIG. 3 shows a block diagram of a processor for identifying a lesion in a biometric image according to embodiments of the present disclosure.

As depicted, in embodiments, the processor 600 may be the processor 111 and 311 shown in FIGS. 1 and 2. The processor 600 may receive a tomography image data from the 3D medical image photographing device 350 to train the machine learning models 211a, 213a, and 230a, and may extract a feature information of an object from based on the received tomography image data.

The tomography image data may be is a feature information data extracted from a three-dimensionally photographed biometric image data or a three-dimensionally photographed biometric image(e.g., CT image, MRI image, etc.) for a specific part(e.g. shoulders, legs, neck, head, etc.) of a patient. In embodiments, the feature information data may be label information for classifying a detected object in the biometric image data. For example, the label may be a category classified into internal organs such as liver, pancreas, and gallbladder expressed in the biometric image, or a category classified as internal tissues such as blood vessels, lymph, and nerves and may be a category classified as a lesion of internal tissue such as fibroadenoma and tumor. In embodiments, the label information may include location information of the object, and the label may be given a weight or order based on the weight and meaning of the object recognized in the biometric image data.

The processor 600 may comprise a data processing unit 210 including a label information generator 211 and a data generator 213.

The label information generator 211 may generate label information corresponding to the received biometric image data using a first machine learning model 211a. The label information may be information on one or more categories according to an object recognized in the received biometric image data. In embodiments, the label information may be stored in the memory unit 113 or the storage device 115 described in FIG. 1 together with information on the biometric image data corresponding to the label information.

The data generator 213 may generate data to input into the feature information model learning unit 230 including the machine learning model 230a. The data generator 213 may generate input data to be input to the third machine learning model 230a and may include lesion size information data or lesion location information data included in the biometric image data received using the second machine learning model 213a, and the lesion size information may be expressed as width and height.

The feature information model learning unit 230 includes the third machine learning model 230a. The data, which includes the image data and the label information, generated and extracted from each of the label information generator 211 and the data generator 213 is input to the third machine learning model 230a. The feature information model learning unit 230 may extract feature information on the biometric image data by performing fusion on the data. The feature information refers to information related to the characteristics of the target image recognized in the biometric image data. For example, the feature information may be the label (e.g., bone tumor) information that classifies objects in biometric image data. If an error occurs in the feature information extracted by the feature information model learning unit 230, a coefficient or a connection weight value used in the fourth machine learning model 230a may be updated.

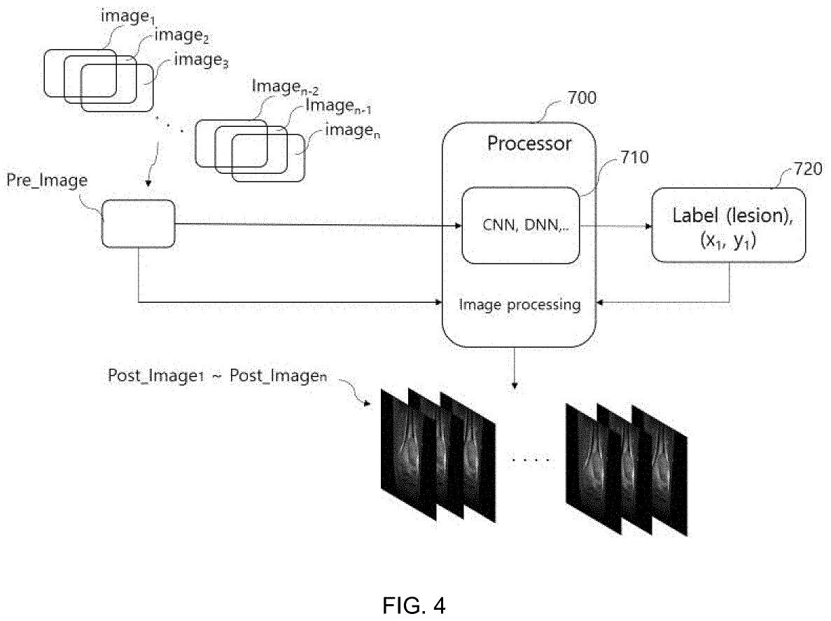

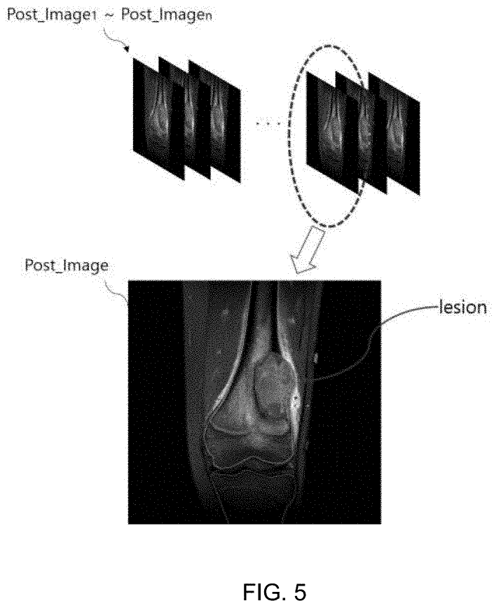

FIG. 4 is a diagram illustratively showing a process of extracting attribute information from tomographic first biometric images and generating second biometric images by a computing device according to an embodiment of the present disclosure. FIG. 5 is a diagram illustratively showing label information areas displayed on second biometric images generated by a computing device according to an embodiment of the present disclosure.

Referring to FIG. 4 and FIG. 5, a plurality of first biometric images (image1, image2, . . . , imagen−1, imagen) such as MRI or CT obtained by 3D tomography of an object are input in the machine learning models 710, and the processor 700 may extract the feature information 720 of the input plurality of first biometric images (hereinafter, referred to as first images) based on the machine learning model 710 included therein. The feature information may be label information for classifying an object (e. g, bone, muscle, etc.) recognized in the biometric image described above, and the label information may include 2-dimensional location information of the object or 2-dimensional size information of the object. etc. Also, the label information may include a lesion such as a bone tumor in a recognized object. The feature information 720 may be stored in the System Memory Unit 113 or the Storage Device 115, the feature information may be image-processed on the first image by the processor 700, and a plurality of second biometric images (Post_images, hereinafter referred to as a second images) may be generated and displayed on the Display Units 130, 330 described in FIG. 1 and FIG. 2 under the control of the processor 700. In this case, the second image may be generated to include a display area corresponding to label information (e. g, lesion).

The processor 700 may calculate the three-dimensional volume (V tumor) of the lesion (e. g, bone tumor) based on the following equation in order to quantify the displayed area (e. g, the lesion information area) in all the second images (Post_images).

$${V}_{tumor} = {\sum }_{i = 1}^{N} {A}_{i} \cdot z = {\sum }_{i = 1}^{N} {n}_{i} s \cdot z \qquad Equation$$

Here, N is a natural number, Aᵢ is an area of the lesion in the i-th second biometric image, nᵢ is the number of pixels for the lesion in the i-th second biometric image, s is a pixel size, z is the thickness of the second biometric image.

Although not shown, the machine learning model 710 may be executed by being input to a computer readable recording medium, may be input to the Memory Unit 113 or Storage Device 115 described in FIG. 1 and FIG. 2, and may be operated and executed by the processor 700.

The extraction of feature information from biometric images can be performed by a computing device, and the computing device is a device that receives a three-dimensionally photographed 3D medical image dataset as learning data, and can generate the data learned as a result of performing the machine learning model. In describing each operation belonging to the method according to the present embodiment, in case that the description of the subject is omitted, it will be able to be understood that the subject of the corresponding operation is the computing device.

As in the above embodiments, it can be clearly understood that the operation and method of the present disclosure can be achieved through a combination of software and hardware or can be achieved only by hardware. Objects of the technical solution of the present invention or parts contributing to the prior art may be implemented in the form of program instructions that can be executed through various computer components and recorded on a machine-readable recording medium. The machine-readable recording medium may include program commands, data files, data structures, etc. alone or in combination. Program instructions recorded on the machine-readable recording medium may be specially designed and configured for the present disclosure, or may be known and usable to those skilled in the art of computer software.

Examples of machine-readable recording media include magnetic media such as hard disks, floppy disks and magnetic tapes, optical recording media such as CD-ROMs and DVDs, and magneto-optical media such as floptical disks and hardware devices specially configured to store and execute program instructions, such as ROM, RAM, flash memory, and the like. Examples of program instructions include high-level language codes that can be executed by a computer using an interpreter as well as machine language codes generated by a compiler.

The hardware device may be configured to act as one or more software modules to perform processing according to the present disclosure and vice versa. The hardware device may be combined with memory such as ROM/RAM for storing program instructions and include a processor such as a CPU or GPU and a communication unit capable of giving and receiving signals with external devices. In addition, the hardware device may include a keyboard, mouse, and other external input devices for receiving commands written by developers.

As described above, the apparatus for quantifying a lesion in a biometric image according to the present invention can quantitatively measure the area of a lesion (e. g, bone tumor) recognized in a 3-dimensional bio image of an object, and thus, the follow-up of the patient according to the medical staffs treatment can be accurately performed. In addition, the apparatus for quantifying a lesion in a biometric image according to the present invention may provide reliable information to medical staff in the evaluation of lesion treatment and determination of recurrence of lesions.

Although the device for quantifying lesions in a bio-image according to the present disclosure is exemplarily applied only to bone tumors in the field of orthopedic surgery for quantitative measurement of lesions, the device has applications recognizing various solid tumors such as brain tumors, neuroblastomas, Wilms' tumors, retinoblastomas, and hepatoblastomas, etc. Furthermore, the device can be applied to any field requiring quantitative measurement of lesions using 3-dimensional medical images.

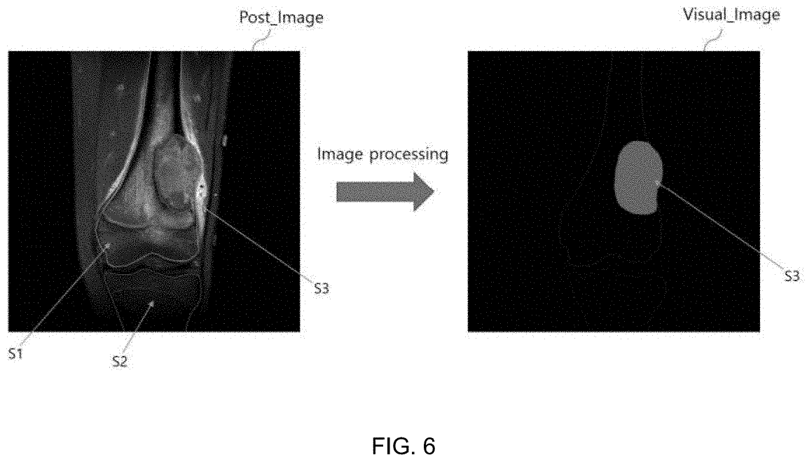

FIG. 6 is a diagram illustrating a third biological image visualizing a label information area in a second biological image generated by a computing device according to an embodiment of the present disclosure.

Referring to FIG. 6, the second biometric image (Post_image) is the second image described in FIG. 4, and is an image generated by image processing by the processor 700 on the first image from which label information, which is attribute information, is extracted. In one embodiment, the second biometric image (Post_image) may be displayed by dividing the bones (bone, S1, S2) and bone tumor (S3), which are label information in the image, but according to the subject of the biometric image (eg, skull, shoulder blade, etc.), label information may be differently partitioned and displayed.

The processor 700 may generate a third biometric image (Visual_image) by performing post image processing so that only the bone tumor region, that is, the lesion region S3, which is the region of interest, among the label information in the second living body image can be visualized. In post-processing, visualization can be performed by adjusting the color, gray level, contrast ratio, etc. of pixels of the lesion area.

The third biometric image (Visual_image) may be stored in the System Memory Unit 113 or the Storage Device 115 described in FIG. 1 and FIG. 2 under the control of the processor 700, and displayed on the Display Unit 130, 330. If the lesion area is visualized in all the three-dimensionally photographed biometric images in the above way, the follow-up observation of the lesion becomes easier for the medical staff, and the evaluation of the treatment of the lesion can be more reliable.

Also, the third biometric image (Visual_image) can be an object that can be used to calculate the 3-dimensional volume of a lesion. That is, the volume of the lesion can be quantitatively calculated by the above-described method by summing only the lesion area visualized in all of the third biometric images.

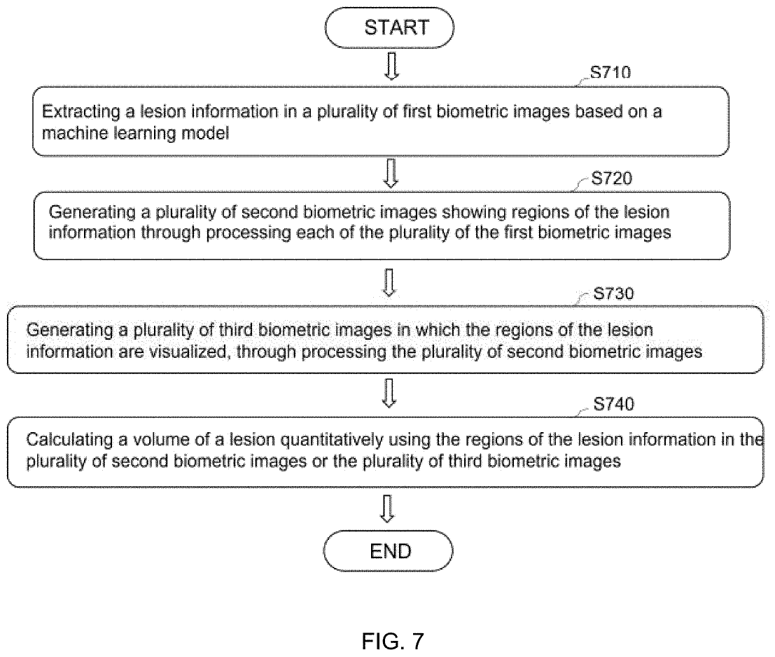

FIG. 7 is a flowchart illustrating a method for quantifying a lesion in a biometric image according to an exemplary embodiment of the present disclosure.

Referring to FIG. 7, the apparatus for quantifying a lesion in a biometric image may use a machine learning model to extract feature information of a plurality of biometric images (a first biometric image) obtained by 3-dimensional tomography. The feature information may be information that can be labeled as a category such as an organ or tissue in the body in the above-described biometric image, but in this embodiment, lesion information will be described as an example. The machine learning model may include, but is not limited to, a Deep Neural Network (DNN), a convolutional neural network (CNN), a Recurrent Neural Network (RNN), etc., which are one of the machine learning algorithms.

In step S710, first biometric images obtained by tomography of the object are input to the machine learning model, and lesion information may be extracted from the input first biometric images based on the machine learning model. Here, the tomography biometric image may correspond to any biometric image obtained by three-dimensionally photographing an organ or tissue inside the human body using a medical imaging device such as MRI or CT. The lesion information may include at least one of presence, size, and location of the lesion, and the location of the lesion may be displayed as a 2D or 3D coordinate value.

In step S720, each of the plurality of first biometric images may be image-processed by the processor of the device for quantifying lesions in the biometric image according to the present disclosure using the extracted lesion information to generate a plurality of second biometric images. In each of the plurality of second biometric images, an area of lesion information may be displayed.

Thereafter, in step S730, the plurality of second biometric images may be image-processed by the processor to generate a plurality of third biometric images so that lesion information regions in the plurality of second biometric images are visualized. At this time, the area of the visualized lesion information can be represented by adjusting the color, gray level, contrast ratio, etc. of a pixel.

Then, in step S740, the volume of the lesion can be quantitatively calculated by the processor using the lesion information area displayed in the plurality of second biometric images or the plurality of third biometric images. Quantitative calculation of the lesion volume can be performed based on the three-dimensional volume calculation formula of the lesion described above.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. A computing device comprising:
a processor; and
a memory that is communicatively coupled to the processor and stores one or more sequences of instructions, which when executed by the processor causes steps to be performed comprising:
extracting a lesion information in a plurality of first biometric images from each of the plurality of first biometric images three-dimensionally photographed of an object based on a machine learning model;
generating a plurality of second biometric images in which a region of the lesion information, by performing image processing on each of the plurality of first biometric images; and
calculating a volume of a lesion quantitatively using the region of the lesion information.

2. The computing device of claim 1,
wherein the volume of the lesion satisfies the following conditional expression:
$${V}_{tumor} = {\sum }_{i = 1}^{N} {A}_{i} \cdot z = {\sum }_{i = 1}^{N} {n}_{i} s \cdot z$$
wherein N is a natural number, Aᵢ is an area of the lesion in the i-th second biometric image, nᵢ is the number of pixels for the lesion in the i-th second biometric image, s is a pixel size, z and is the thickness of the second biometric image.

3. The computing device of claim 1,
wherein the lesion information includes at least one of a size of the lesion and a location of the lesion.

4. The computing device of claim 1,
wherein the lesion is a solid tumor including a bone tumor.

5. The computing device of claim 1,
wherein the processor performs image processing on the lesion information in the plurality of second biometric to generate a plurality of third biometric images in which only the region of the lesion information is visualized.

6. A method for quantifying a lesion in a biometric image, comprising:
extracting a lesion information in a plurality of first biometric images from each of the plurality of first biometric images three-dimensionally photographed of an object based on a machine learning model;
generating a plurality of second biometric images in which a region of the lesion information, by performing image processing on each of the plurality of first biometric images; and
calculating a volume of the lesion quantitatively using the region of the lesion information.

7. The method of claim 6,
wherein the lesion information includes at least one of a size of the lesion and a location of the lesion.

8. The method of claim 6, further comprising,
performs image processing on the lesion information in the plurality of second biometric to generate a plurality of third biometric images in which only the region of the lesion information is visualized.
